(19) United States
(12) Patent Application Publication
(10) Pub. No.: US 2015/0011755 A1
(43) Pub. Date: Jan. 8, 2015

(54) AMINE SALTS OF PROSTAGLANDIN ANALOGS

(71) Applicant: DR. REDDYS LABORATORIES LIMITED, Hyderabad, AP (IN)

(72) Inventor: Mark P Jackson, Cambridge (GB)

(73) Assignee: DR. REDDYS LABORATORIES LIMITED, Hyderbad (IN)

(21) Appl. No.: 14/375,013

(22) PCT Filed: Feb. 6, 2013
(86) PCT No.: PCT/IB2013/050976

§ 371 Date: Jul. 28, 2014

Related U.S. Application Data
(60) Provisional application No. 61/595,726, filed on Feb. 7, 2012.

(51) Int. Cl. C07C 67/475; C07C 69/732; C07D 311/94; C07C 211/07; C07C 211/03; C07D 295/027; C07C 211/09; C07C 211/35; C07C 211/06; C07C 211/04; C07C 69/736; C07C 211/05

(52) U.S. Cl. CPC C07C 67/475; C07C 69/736; C07C 69/732; C07D 311/94; C07C 211/07; C07C 211/05; C07D 295/027; C07C 211/09; C07C 211/35; C07C 211/06; C07C 211/04; C07C 211/03
USPC 544/106; 560/62; 560/75; 549/396

(57) ABSTRACT

The present application relates to amine salts of prostaglandin analogs and their uses for the preparation of substantially pure prostaglandin analogs. Specific embodiments relate to amine salts of tafluprost and their uses for the preparation of substantially pure tafluprost.

### INTRODUCTION

Aspects of the present application relate to amine salts of prostaglandin analogs such as tafluprost, bimatoprost, latanoprost, lubiprostone etc. Further aspects relate to the use of amine salts of prostaglandin analogs as intermediates in the preparation of substantially pure prostaglandin analogs such as tafluprost, bimatoprost, latanoprost, lubiprostone etc.

Prostaglandin analogs, including tafluprost, bimatoprost, and latanoprost, are useful for treating glaucoma, and lubiprostone is useful for treating chronic idiopathic constipation and irritable bowel syndrome.

The synthesis of prostaglandin analogs in pure form is known to be difficult, because of their complex structure. However, it is important to synthesize the prostaglandin analogs in a very pure form so that they can be used as active pharmaceutical ingredients.

European Patent No. 8509621 discloses a process for the preparation of tafluprost. In the first step, (3aR,4R,5R,6aS)-4-formyl-2-oxohexahydro-2H-cyclopenta[b]furan-5-ylbenzoate (CTAF 1(i)) is condensed with dimethyl (2-oxo-3-phenoxypropyl)-phosphonate in the presence of lithium chloride and triethylamine, to provide (3aR,4R,5R,6aS)-2-oxo-4-((E)-3-oxo-4-phenoxybut-1-en-1-yl)hexahydro-2H-cyclopenta[b]-furan-5-ylbenzoate (CTAF1). In the second step, CTAF 1 is reacted with morpholinosulfurtrifluoride to provide (3aR,4R,5R,6aS)-4-((E)-3,3-difluoro-4-phenoxybut-1-en-1-yl)-2-oxohexahydro-2H-cyclopenta-[b]furan-5-yl benzoate (CTAF2). CTAF 2 is debenzoylated by potassium carbonate in methanol, to provide (3aR,4R,5R,6aS)-4-((E)-3,3-difluoro-4-phenoxybut-1-en-1-yl)-5-hydroxyhexahydro-2H-cyclopenta[b]furan-2-one(CTAF 3), which is further reduced by diisobutyl aluminum hydride (DIBALH) to provide (3aR,4R,5R,6aS)-4-((E)-3,3-difluoro-4-phenoxybut-1-en-1-yl) hexahydro-2H-cyclopenta[b]furan-2,5-diol (CTAF 4). CTAF 4 is then treated with (4-carboxybutyl)triphenylphosphonium bromide, in the presence of potassium bis(trimethylsilyl)amide in THF, to provide (Z)-7-((1 R,2R,3R,5S)-2-((E)-3,3-difluoro-4-phenoxybut-1-en-1-yl)-3,5-dihydroxycyclopentyl)hept-5-enoic acid (“tafluprost free acid,” CTAF5), which is reacted with isopropyl iodide in the presence of DBU to provide (Z)-isopropyl 7-((1R,2R,3R,5S)-2-((E)-3,3-difluoro-4-phenoxybut-1-en-1-yl)-3,5-dihydroxycyclopentyl)hept-5-enoate (“tafluprost,” CTAF 6). The reaction sequence is summarized in Scheme 1.

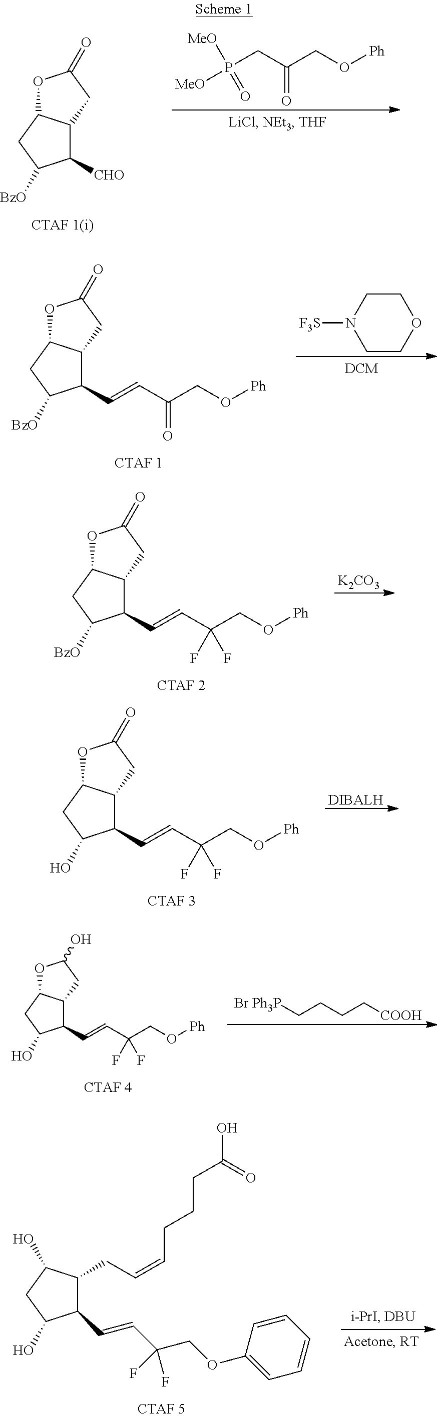

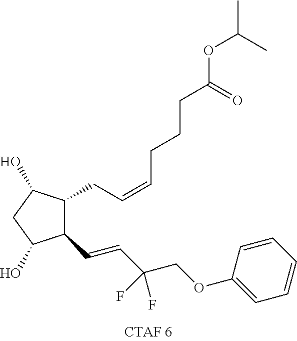 [SMILES: CC(C)OC(=O)CCC/C=C\C[C@@H]1[C@@H](/C=C/C(F)(F)COc2ccccc2)[C@H](O)C[C@@H]1O]

U.S. Patent Application Publication No. 2010/0105775A1 discloses amino acid salts of prostaglandins. The application also discloses a process for the preparation of prostaglandins, comprising forming an amino acid salt of a prostaglandin and converting the amino acid salt to the prostaglandin.

A need remains for improved processes to make prostaglandin analogs.

### SUMMARY

An aspect of the present application relates to amine salts of prostaglandin analogs such as tafluprost, bimatoprost, latanoprost, and lubiprostone.

An aspect of the present application relates to uses of amine salts of prostaglandin analogs for the preparation of substantially pure prostaglandin analogs.

### BRIEF DESCRIPTION OF THE DRAWING

FIG. 1 shows a powder X-ray diffraction (PXRD) pattern of a dicyclohexylamine salt of tafluprost, obtained using the procedure of Example 6.

### DETAILED DESCRIPTION

An aspect of the present application relates to processes for the preparation of substantially pure prostaglandin analogs such as tafluprost, bimatoprost, latanoprost, and lubiprostone. A specific aspect of the present application relates to the preparation of tafluprost. Another aspect of the present application relates to processes for the preparation of pure tafluprost, substantially free from impurities, specifically the trans-isomer of tafluprost.

It has now been found that an amine salt of prostaglandin analog is a solid compound that can be easily purified by crystallization. Prostaglandin analogs prepared through an amine salt of the prostaglandin analogs are generally more pure than prostaglandin analogs prepared directly, without using amine salts of prostaglandin analogs.

Tafluprost will be used in the following discussion as a representative of the prostaglandin analogs, to simplify the description of the processes. However, the general techniques are also applicable to the other analogs and the scope of the disclosure is not to be limited to tafluprost.

In a first step for the synthesis of substantially pure tafluprost, the compound (3aR,4R,5R,6aS)-4-formyl-2-oxohexahydro-2H-cyclopenta[b]furan-5-yl benzoate (CTAF 1(i)) is condensed with dimethyl (2-oxo-3-phenoxypropyl)-phosphonate, in the presence of a base and a zinc compound, to form CTAF 1. The base may be any base known in the art. In embodiments, the base is an alkali metal hydride such as sodium hydride, potassium hydride, or lithium hydride. Examples of useful zinc compounds include zinc chloride, zinc iodide, zinc sulfate, and zinc nitrate. The reaction may be performed in the presence of a solvent, such as an aprotic solvent. Examples of useful aprotic solvents include, without limitation thereto, N,N-dimethylformamide (DMF), dimethylsulfoxide, tetrahydrofuran (THF), N,N-dimethylacetamide and acetonitrile.

CTAF 1 prepared by the above described reaction is treated with any fluorinating agent known in the art. In embodiments, the fluorinating agent is diethylaminosulfurtrifluoride. The reaction may be carried out in a halogenated hydrocarbon solvent, to provide CTAF 2. Useful halogenated hydrocarbon solvents include dichloromethane (DCM), chloroform, and carbon tetrachloride.

CTAF 2 is treated with a hydride reagent in a hydrocarbon solvent to provide CTAF 4. Useful hydride reagents include sodium hydride, potassium hydride, and diisobutyl aluminum hydride. Solvents that may be used for the reaction include aliphatic and aromatic hydrocarbon solvents. Specific useful solvents include benzene and toluene.

CTAF 4 undergoes a Wittig reaction with (4-carboxybutyl)triphenylphosphonium bromide to provide CTAF 5. The base used for the reaction may be an alkali metal alkoxide or an amide base. Useful bases include sodium methoxide, sodium ethoxide, potassium tert-butoxide, sodium bis(trimethylsilyl)amide (NaHMDS), potassiumbis(trimethylsilyl)amide (KHMDS), lithium bis(trimethylsilyl)amide (LiHMDS), sodamide, lithium diisopropylamide, and n-butyl lithium. The solvent used as the medium for the reaction may be any aprotic solvent. Specific useful aprotic solvents include tetrahydrofuran, toluene, benzene, dimethylsulfoxide, N,N-dimethylacetamide, acetonitrile, and N,N-dimethylformamide.

An aspect of the present application relates to amine salts of prostaglandin analogs such as tafluprost, bimatoprost, latanoprost, and lubiprostone. Amine salts of prostaglandin analogs are in general solid compounds and thus may be purified by crystallization methods. Examples of useful amines for salt formation include, but are not limited to, tert-butylamine, diethylamine, dibutylamine, morpholine, 3-dimethylamino-1-propylamine, dicyclohexylamine (DCHA), diisopropylamine, N-tert-butylbenzylamine, N-benzylmethylamine, (R)-α-methylbenzylamine, (S)-α-methylbenzylamine, benzylamine, dibenzylamine, cyclohexylamine and tert-octylamine.

It has been observed that when tafluprost is prepared using a dicyclohexylamine(DCHA) salt of tafluprost as an intermediate, the trans-isomer level in the final product may be controlled to be within desired levels. The Wittig reaction of CTAF 4 with (4-carboxybutyl)triphenylphosphonium bromide to provide CTAF 5 produces trans-isomer up to a level of 1%. The removal of trans-isomer was found to be very difficult using general purification methods like crystallization and chromatography. The removal of the trans-isomer was successful to some extent by preparative HPLC methods. However, the use of preparative HPLC methods is not feasible for large scale manufacturing of an active pharmaceutical ingredient.

An aspect of the present application relates to amine salts of prostaglandin analogs, wherein the salts are solid in nature. Another aspect of the present application relates to amine salts of tafluprost, wherein the salts are solid in nature. Another aspect of the present application relates to a DCHA salt of tafluprost. Still another aspect of the present application relates to a DCHA salt of tafluprost having less than about 0.2% of trans-isomer, or less than about 0.15% of trans-isomer, or less than about 0.1% of trans-isomer.

In embodiments, CTAF 5 is dissolved in an organic solvent and treated with dicyclohexylamine for sufficient time to produce the DCHA salt of tafluprost. The salt is isolated and, optionally, crystallized from an organic solvent. The organic solvents used for the reaction and crystallization may be any solvents known in the art. The solvents used as the reaction medium and for crystallization may be the same solvent or different solvents. Useful solvents include ethers, esters, ketones, and hydrocarbons. Specific ketone solvents include, without limitation, acetone, methyl isobutyl ketone, methyl ethyl ketone, methyl isopropyl ketone, cyclopetanone, and butanone.

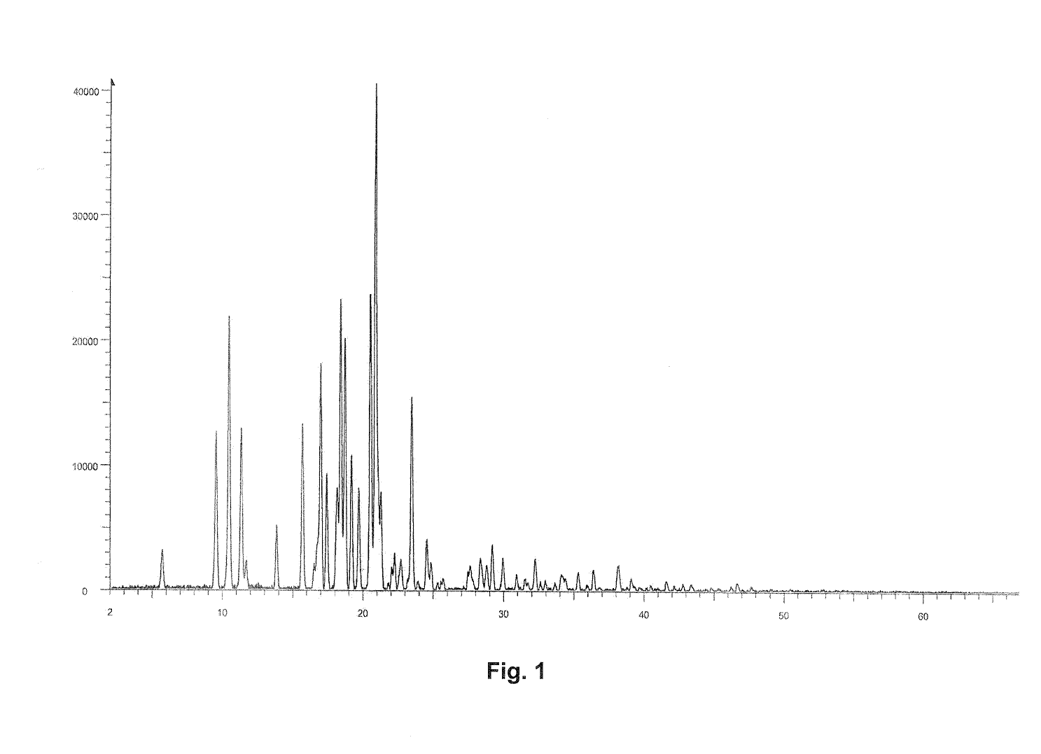

An aspect of the present application relates to a crystalline dicyclohexylamine salt of tafluprost. Another aspect of the present application relates to a crystalline dicyclohexylamine salt of tafluprost characterized by its powder X-ray diffraction (PXRD) pattern having peaks at about 20.85, 20.46, 18.34 and 15.64±0.2 degrees 2θ. Still another aspect of the present application relates to a crystalline dicyclohexylamine salt of tafluprost characterized by its powder X-ray diffraction (PXRD) pattern having additional peaks at about 5.64, 9.48, 10.40 and 11.27±0.2 degrees 2θ. Yet another aspect of the present application relates to a crystalline dicyclohexylamine salt of tafluprost characterized by its powder X-ray diffraction (PXRD) pattern having additional peaks at about 13.78 and 16.93±0.2 degrees 2θ. Another aspect of the present application relates to a crystalline dicyclohexylamine salt of tafluprost characterized by its powder X-ray diffraction (PXRD) pattern which is substantially similar to FIG. 1.

An aspect of the present application relates to the use of amine salts of prostaglandin analogs for the preparation of substantially pure prostaglandin analogs. Another aspect of the present application relates to the use of amine salts of tafluprost for the preparation of substantially pure tafluprost, substantially free from impurities and specifically from the trans-isomer of tafluprost. Another aspect of the present application relates to the use of a DCHA salt of tafluprost for the preparation of substantially pure tafluprost, substantially free from any impurity and specifically from the trans-isomer of tafluprost. Another aspect of the present application relates to processes for the preparation of tafluprost having less than about 0.2% of trans-isomer, or less than about 0.15% of trans-isomer, or less than about 0.1% of trans-isomer.

The amine salt of a prostaglandin analog may be treated with an acid in an organic solvent to produce the prostaglandin analog free acid. The acid may be any organic or mineral acid. In embodiments, the acid is a mineral acid such as hydrochloric acid, sulfuric acid, nitric acid, hydrofluoric acid, perchloric acid, boric acid, or phosphoric acid. In embodiments, the organic solvent is a water immiscible organic solvent, such as an ether, ester, hydrocarbon, or chlorinated hydrocarbon. An example of a useful solvent is ethyl acetate. The free acid of tafluprost may be converted to tafluprost by treating it with isopropyl iodide in acetone in presence of a base, like, 1,8-diazabicyclo[5.4.0]undec-7-ene (DBU), as disclosed in European Patent 850926B1.

Aspects of the present application also relate to pharmaceutical compositions comprising prostaglandin analogs such as tafluprost. The pharmaceutical compositions may be in the form of ophthalmic solutions, ophthalmic suspensions, ophthalmic ointments, ophthalmic gels, ophthalmic inserts, and the like. Compositions may contain any number of pharmaceutically acceptable excipients, including: surfactants such as polysorbate 80, polyoxyl 40 stearate, and the like; buffering agents such as sodium phosphate, sodium acetate, and the like; stabilizers such as disodium edetate, and the like; tonicity agents such as sodium chloride, and the like; preservatives such as benzalkonium chloride, methyl paraben, and the like; penetration enhancers such as bile acid salts, and the like; vehicles such as water, and the like; antioxidants such as ascorbic acid, glutathione, ubiquinol, and the like; polymers such as polyacrylic acid, and the like; and viscosity modifiers such as a carbomer, polyvinyl alcohol, etc.

Certain specific aspects and embodiments are further described by the following examples, being provided only for purposes of illustration, and the scope of the disclosure is not intended to be limited by the examples.

### EXAMPLE 1

### Preparation of CTAF 1

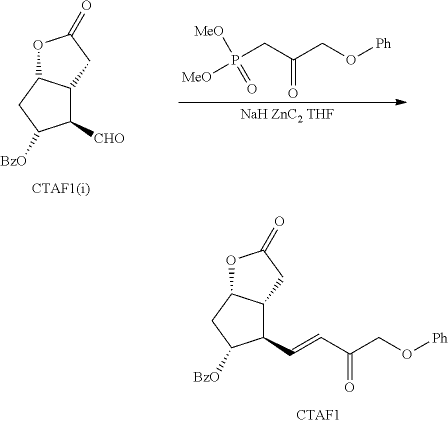

To a stirred suspension of sodium hydride (60% dispersion in mineral oil, 0.217 g, 5.429 mmol) in THF (5 mL) was added a solution of dimethyl (2-oxo-3-phenoxypropyl)phosphonate (1.21 g, 4.705 mmol) in THF (2 mL), over 15 minutes at 0-5° C. under a nitrogen atmosphere. The mixture was warmed to 25-35° C., 0.5 M zinc chloride solution in THF (9.4 mL, 4.705 mmol) was added over 10 minutes, and then the mixture was stirred for 15 minutes at 25-35° C. CTAF1(i) (3aR,4R,5R,6aS)-4-formyl-2-oxohexahydro-2H-cyclopenta[b]furan-5-yl benzoate (1 g) in dichloromethane (10 mL) was added over 5 minutes at 25-35° C. The temperature was raised to 35-40° C. and the mixture was stirred for 2 hours under a nitrogen atmosphere. The mixture was cooled to 15° C. and the reaction was quenched by adding acetic acid (0.2 mL), followed by adding saturated ammonium chloride solution (10 mL), and further stirring for 15 minutes. The organic layer was separated and the aqueous layer was extracted with ethyl acetate (5 mL). The combined organic layers were evaporated under reduced pressure below 50° C. The crude product was purified by column chromatography on silica gel (100-200 mesh) with 30% ethyl acetate in hexane, to afford the title compound (0.9 g, 61% yield).

### EXAMPLE 2

### Preparation of CTAF 2

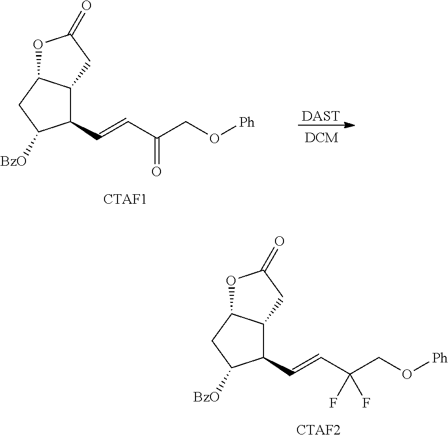

To a stirred solution of CTAF1 (5 g, 0.0123 mol) in dichloromethane (100 mL) was added diethylaminosulfurtrifluoride (13 mL, 0.09841 mol) at 0-5° C. under a nitrogen atmosphere. The temperature was raised to 25-35° C. and maintained for 24 hours under a nitrogen atmosphere at the same temperature. The mass was slowly added into a saturated sodium bicarbonate solution (75 mL) at 0-5° C. Temperature was raised to 25-35° C., the layers were separated, and the aqueous layer was extracted with dichloromethane (2×25 mL). The combined organic layer was washed with water (25 mL) and dried over sodium sulfate (5 g). The organic layer was evaporated to dryness under reduced pressure below 40° C. The crude product was purified by column chromatography on silica gel (100-200 mesh) with 30% ethyl acetate in hexane, to afford the title compound (4.2 g, 79% yield).

### EXAMPLE 3

### Preparation of CTAF 4

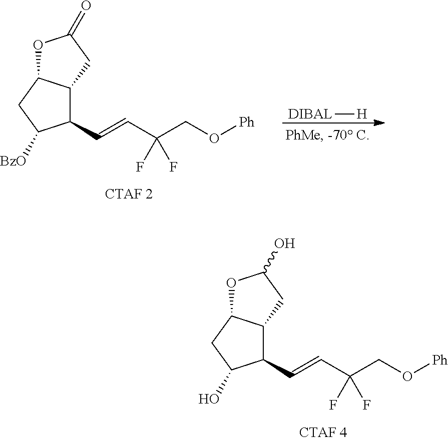

CTAF 2 (2.30 g, 5.37 mmol) was dissolved in toluene (25 mL) and the solution was cooled to −65° C. under nitrogen. Diisobutyl aluminum hydride (1.5 M in toluene, 11.8 mL, 17.7 mmol) was added over 15 minutes at −61 to −65° C. The mixture was stirred for 3 hours and then the reaction was quenched by adding methanol (1.5 mL). Sulfuric acid (1 M, 25 mL) was added and the temperature rose to −20° C. during the addition. Methyl t-butyl ether (MTBE) (10 mL) was added and the mixture was allowed to warm to room temperature. The organic phase was separated and the aqueous phase was extracted with MTBE (2×10 mL). The combined organic phase was washed with water (10 mL), saturated aqueous sodium bicarbonate (10 mL), and then brine (10 mL). The washes were back-extracted with MTBE (10 mL). The combined organic phases were dried with magnesium sulfate, filtered, and evaporated to give a colourless oil (2.20 g). The crude product was chromatographed on silica (60 g), eluting with a mixture of ethyl acetate and heptane (2:1 by volume), and then with ethyl acetate, to give CTAF 4 as a colourless oil (1.71 g, 97% yield).

### EXAMPLE 4

### Preparation of CTAF 2

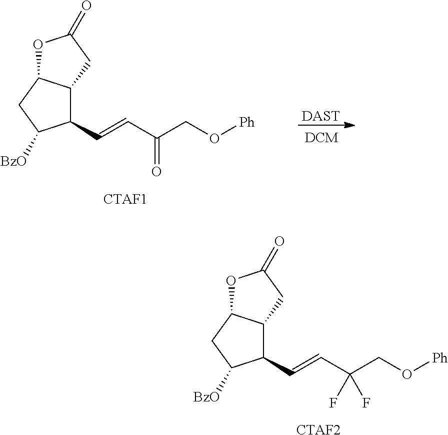

To a stirred solution of CTAF1 (20 g, 0.0492 mol) in dichloromethane(400 mL) was added diethylaminosulfurtrifluoride (52 mL, 0.393 mol) at 0-10° C. under a nitrogen atmosphere. The temperature was raised to 25-35° C. and maintained for 96 hours under a nitrogen atmosphere at that temperature. The mass was slowly added to a saturated NaHCO₃ solution (600 mL) at 0-10° C. The mixture was heated to 25-35° C. and filtered through a Celite bed. The layers were separated and the aqueous layer was extracted with DCM (2×100 mL). The combined organic layer was washed with 10% NaCl solution (100 mL) and evaporated to dryness under reduced pressure below 40° C. The residue was purified by column chromatography on silica gel (100-200 mesh) with 30% ethyl acetate in hexane.

Column purified material was dissolved in MTBE (80 mL) at 40° C. and stirred for 30 minutes at that temperature. Diisopropyl ether (160 mL) was added at 35-40° C. and stirring continued for 30 minutes at 35-40° C. Cooled the mass to 5-15° C. and stirred for 30 minutes at that temperature. The solid was filtered, washed with a mixture of MTBE and diisopropyl ether (DIPE) (1:2 by volume, 60 mL), and dried at 40° C. under vacuum, to afford pure CTAF2 (12.0 g, 57% yield).

### EXAMPLE 5

### Preparation of CTAF 5

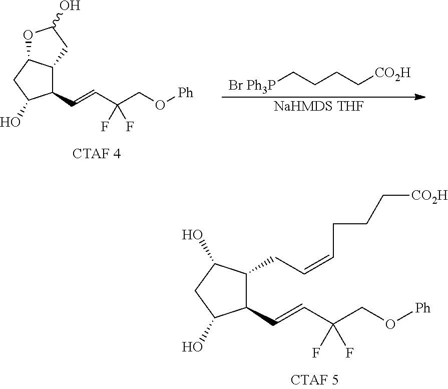

(4-Carboxybutyl)triphenylphosphonium bromide (10.32 g, 23.3 mmol, 4 eq) was suspended in THF (20 mL) under a nitrogen atmosphere and cooled to 5° C. NaHMDS solution (1M in THF, 46.6 mL, 46.6 mmol, 8 eq) was added over 10 minutes. The red/orange mixture was stirred for 30 minutes. A solution of CTAF 4 (1.90 g, 5.82 mmol) in THF (10 mL) was added over 30 minutes at 0-3° C. The mixture was stirred for 1.5 hours and then the reaction was quenched by adding water (30 mL) and the mass was warmed to room temperature. The aqueous phase was separated and the organic phase was washed with water (20 mL). The combined aqueous phases were washed with MTBE (30 mL). The organic phases up to this point were discarded.

The aqueous phase was acidified with 2M hydrochloric acid (14 mL, to pH 3-4) and extracted with ethyl acetate (2×30 mL). The combined ethyl acetate layers were washed with brine (20 mL), dried with magnesium sulfate, filtered, and evaporated under reduced pressure to give CTAF 5 asa yellow oil (8.60 g).

A 2.96 g sample was removed and the remainder (5.64 g) was chromatographed on silica (30 g) eluting with ethyl acetate to give purified CTAF 5 (1.41 g) asa yellow oil. NMR analysis showed approximately 90% purity, remainder triphenyl phosphine oxide.

### EXAMPLE 6

### Preparation of CTAF 5 DCHA Salt

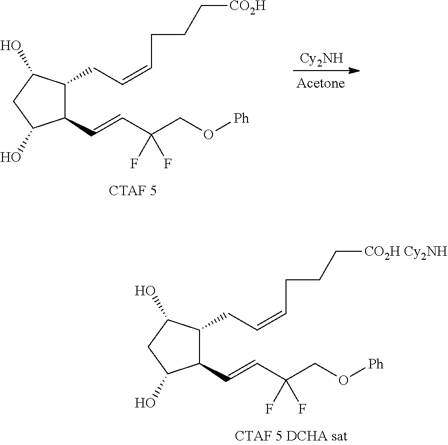

CTAF5 (11.72 g, 90% purity, 25.7 mmol, containing 1.4% trans isomer) was dissolved in acetone (60 mL). Dicyclohexylamine (4.66 g, 25.7 mmol) was added and the mixture was stirred at room temperature overnight. The solid was filtered and washed with acetone (6 mL), then dried to give the DCHA salt (12.93 g, 85% yield, 0.29% trans-isomer).

A sample (7.03 g) was further purified by recrystallisation. It was dissolved in hot acetone (30 mL) and cooled to room temperature with stirring. The mixture was stirred for 3 hours, filtered and the solid was washed with acetone (3 mL) and dried to give a white solid (6.41 g, 91% recovery, 0.11% trans-isomer).

A PXRD pattern of the product is shown as FIG. 1, obtained using copper Kα radiation. In the drawing, the y-axis is intensity units and the x-axis is the 2-theta angle, in degrees.

### EXAMPLE 7

### Preparation of CTAF 6

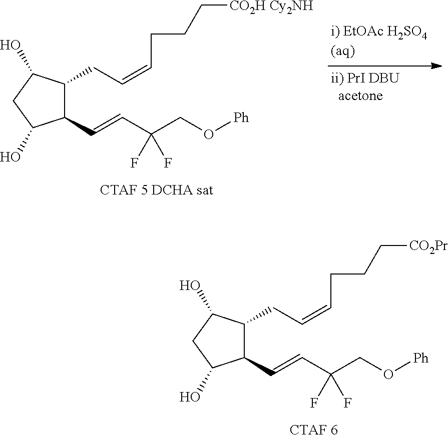

CTAF 5 DCHA salt (5.80 g, 9.80 mmol) was suspended in ethyl acetate (20 mL). Sulfuric acid (1 M, 20 mL) was added and the mixture was stirred until a clear solution was obtained. The organic phase was separated and the aqueous phase was extracted with ethyl acetate (2×20 mL). The combined organic layers were washed with water (15 mL) and brine (15 mL), dried with magnesium sulfate, filtered, and evaporated. The residue was dissolved in acetone (40 mL) and charged into a jacketed vessel at 30° C. 1,8-Diazabicyclo[5.4.0]undec-7-ene (DBU) (8.95 g, 58.8 mmol) was added, then 2-iodopropane (10.0 g, 58.8 mmol) was added, and the mixture was stirred for 20 hours. The mixture was concentrated under reduced pressure and the residue was partitioned between ethyl acetate (30 mL) and aqueous potassium dihydrogen orthophosphate (8 g) in water (50 mL). The organic phase was separated and the aqueous was extracted with ethyl acetate (30 mL). The combined organic phases were washed with brine (20 mL), dried with magnesium sulfate, filtered and evaporated to give a yellow oil (4.83 g). The crude product was chromatographed on silica (130 g), eluting with a mixture of ethyl acetate and heptane (2:1 by volume), to give CTAF 6 (3.98 g, 90% yield) as a colorless oil.

1. An amine salt of a prostaglandin analog.

2. The compound of claim 1, wherein the prostaglandin analog is selected from the group of tafluprost, bimatoprost, latanoprost, and lubiprostone.

3. The compound of claim 1, wherein the amine is selected from a group of tert-butylamine, diethylamine, dibutylamine, morpholine, 3-dimethylamino-1-propylamine, dicyclohexylamine (DCHA), diisopropylamine, N-tert-butylbenzylamine, N-benzylmethylamine, (R)-α-methylbenzylamine, (S)-α-methylbenzylamine, benzylamine, dibenzylamine, cyclohexylamine and tert-octylamine.

4. The compound of claim 1, wherein the prostaglandin analog is tafluprost.

5. The compound of claim 1, wherein the amine is dicyclohexylamine.

6. An amine salt of tafluprost.

7. The compound of claim 6, wherein the amine is selected from a group of tert-butylamine, diethylamine, dibutylamine, morpholine, 3-dimethylamino-1-propylamine, dicyclohexylamine (DCHA), diisopropylamine, N-tert-butylbenzylamine, N-benzylmethylamine, (R)-α-methylbenzylamine, (S)-α-methylbenzylamine, benzylamine, dibenzylamine, cyclohexylamine and tert-octylamine.

8. The compound of claim 6, wherein the amine is dicyclohexylamine.

9. A crystalline dicyclohexylamine salt of tafluprost.

10. The crystalline dicyclohexylamine salt of tafluprost of claim 9 having an X-ray powder diffraction pattern with peaks at about 20.85, 20.46, 18.34 and 15.64±0.2 degrees 2θ.

11. A process of preparing a prostaglandin analog which method comprises crystallization of an amine salt of prostaglandin analog.

12. The process as claimed in claim 11, wherein the prostaglandin analog is selected from the group of tafluprost, bimatoprost, latanoprost, and lubiprostone.

13. The process as claimed in claim 11, wherein the prostaglandin analog is tafluprost.

14. The process as claimed in claim 11, wherein the amine is selected from a group of tert-butylamine, diethylamine, dibutylamine, morpholine, 3-dimethylamino-1-propylamine, dicyclohexylamine (DCHA), diisopropylamine, N-tert-butylbenzylamine, N-benzylmethylamine, (R)-α-methylbenzylamine, (S)-α-methylbenzylamine, benzylamine, dibenzylamine, cyclohexylamine and tert-octylamine.

15. The process as claimed in claim 11, wherein the amine salt is dicyclohexylamine.

16. A process of preparing tafluprost, which method comprises crystallization of dicyclohexylamine salt of tafluprost.

17. A process for the preparation of dicyclohexylamine salt of tafluprost, comprising the steps of:
i) condensation of (3aR,4R,5R,6aS)-4-formyl-2-oxohexahydro-2H-cyclopenta[b]furan-5-yl benzoate (CTAF 1(i)) with dimethyl (2-oxo-3-phenoxypropyl)-phosphonate in the presence of a base and a zinc compound to form (3a R,4R,5R,6aS)-2-oxo-4-((E)-3-oxo-4-phenoxybut-1-en-1-yl)hexahydro-2H-cyclopenta[b]furan-5-yl benzoate (CTAF 1);
ii) Treatment of CTAF 1 with a fluorinating agent in a halogenated hydrocarbon solvent to provide difluoro compound, (3aR,4R,5R,6aS)-4-((E)-3,3-difluoro-4-phenoxybut-1-en-1-yl)-2-oxohexahydro-2H-cyclopenta[b]furan-5--yl benzoate (CTAF 2);
iii) Treatment of CTAF 2 with a hydride reagent in a hydrocarbon solvent to provide (3aR,4R,5R,6aS)-4-((E)-3,3-difluoro-4-phenoxybut-1-en-1-yl)hexahydro-2H-cyclopenta[b]-furan-2,5-diol (CTAF 4);
iv) reaction of CTAF 4 in the presence of a base and an organic solvent, to provide (Z)-7-((1R,2R,3R,5S)-2-((E)-3,3-difluoro-4-phenoxybut-1-en-1-yl)-3,5-dihydroxycyclopentyl)-hept-5-enoic acid (CTAF 5);
v) treating CTAF 5 with an dicyclohexylamine in an organic solvent to provide an amine salt of CTAF 5;
vi) optionally, crystallizing the amine salt of CTAF 5 from an organic solvent.

18. The process of claim 17, wherein the base in step i) is an alkali metal hydride.

19. The process of claim 17, wherein the zinc compound in step i) is selected from a group of zinc chloride, zinc iodide, zinc sulfate, and zinc nitrate.

20. The process of claim 17, wherein the fluorinating agent in step ii) is diethylaminosulfurtrifluoride.

21. The process of claim 17, wherein the hydride reagent in step iii) is selected from a group of sodium hydride, potassium hydride, and diisobutyl aluminum hydride.

22. The process of claim 17, wherein the base in step (iv) is an amide base.

23. The process of claim 17, wherein the base in step (iv) is selected from a group of sodium (trimethylsilyl)amide, potassium (trimethylsilyl)amide, lithium (trimethylsilyl)-amide, sodamide, lithium diisopropylamide.

24. The process of claim 17, wherein the solvent in step (iv) is tetrahydrofuran, toluene, benzene, dimethylsulfoxide, dimethylacetamide, acetonitrile, N,N-dimethylformamide.

25. The process of claim 17, wherein the solvent in step (v) is selected from a group of ethers, esters, ketones, hydrocarbons.

26. The process of claim 17, wherein the solvent for crystallization in step (vi) is selected from a group of ethers, esters, ketones, hydrocarbons.

27. The process of claim 17, wherein the solvent in step (v) and step (vi) is a ketone.

28. The process of claim 17, wherein the solvent in step (v) and step (vi) is acetone.

29. The process of claim 17, further comprising conversion of dicyclohexylamine salt of tafluprost to tafluprost.

30. The process of claim 29, comprising the steps of:
i) treatment of dicyclohexylamine salt of tafluprost with an acid;
ii) treatment of the product of step i) with isopropyl iodide in presence of a base.

31. The process of claim 30, wherein the base in step ii) is 1,8-diazabicyclo-[5.4.0]undec-7-ene (DBU).
